(19) United States
(12) Patent Application Publication
(10) Pub. No.: US 2025/0262479 A1
(43) Pub. Date: Aug. 21, 2025

(54) EXERCISE ANALYSIS METHOD, SERVER AND TERMINAL APPARATUS USED FOR EXERCISE ANALYSIS

(71) Applicant: Wistron Corporation, New Taipei City (TW)

(72) Inventors: Yi Yun Hsieh, New Taipei City (TW); Ming Jie Li, New Taipei City (TW); Yu Hsien Chen, New Taipei City (TW)

(73) Assignee: Wistron Corporation, New Taipei City (TW)

(21) Appl. No.: 18/613,082

(22) Filed: Mar. 21, 2024

(30) Foreign Application Priority Data
Feb. 19, 2024 (TW) 113105777

(51) Int. Cl. A63B 24/00

(52) U.S. Cl. CPC A63B 24/0003; A63B 24/0062; A63B 2024/0071; A63B 2225/50

(57) ABSTRACT

An exercise analysis method, a server, and a terminal apparatus used for exercise analysis are disclosed. In the method, sensing data is transmitted through a network, analysis information is generated according to the sensing data, the analysis information is given feedback through the network, and the analysis information is presented by a user interface. The sensing data corresponds to a motion state. The analysis information is used to analyze the motion state. In this way, the operating experience may be improved.

### CROSS-REFERENCE TO RELATED APPLICATION

This application claims the priority benefit of Taiwan application serial no. 113105777, filed on Feb. 19, 2024. The entirety of the above-mentioned patent application is hereby incorporated by reference herein and made a part of this specification.

### BACKGROUND

### Technical Field

This disclosure relates to an analysis technology, and in particular to an exercise analysis method, a server and a terminal apparatus used for exercise analysis.

### Description of Related Art

In addition to patients with instinctive compensatory responses due to disease or degeneration, postural training is also required for athletes who need to improve their posture. In the early days, training was mostly based on the experience of physiotherapists and their paper records. However, as the physiotherapist's experience varies, different diagnoses or treatments may be given to subjects with the same cause of disease. As a result, patients may need some adjustment period due to change of physiotherapist. Although sensors can be used to track body movements in the recent times, specialized personnel are required to set up and operate the equipment in special environments.

### SUMMARY

The disclosure provides an exercise analysis method, a server and a terminal apparatus used for exercise analysis, capable of reducing difficulty of operation and is suitable for the general public.

The exercise analysis method in the embodiment of the disclosure includes (but is not limited to) the following. Sensing data is transmitted through a network. Analysis information is generated according to the sensing data, and the analysis information is presented by a user interface. The sensing data corresponds to a motion state. The analysis information is used to analyze the motion state.

The server used for exercise analysis in the embodiment of the disclosure includes (but is not limited to) a communication transceiver, a storage, and a processor. The storage stores a program code. The processor is coupled to the communication transceiver and the storage. The processor loads the program code and executes receiving sensing data through a network by the communication transceiver; generating analysis information according to the sensing data; transmitting analysis information through the network by the communication transceiver. The sensing data corresponds to a motion state. The analysis information is used to analyze the motion state. The analysis information is used to be presented by a user interface.

The terminal apparatus used for exercise analysis in the embodiment of the disclosure includes (but is not limited to) a display, a communication transceiver, a storage, and a processor. The storage stores a program code. The processor is coupled to the display, the communication transceiver, and the storage. The processor loads the program code and executes transmitting sensing data through a network by the communication transceiver; receiving analysis information through the network by the communication transceiver; presenting the analysis information on a user interface by the display. The sensing data corresponds to a motion state. The analysis information is generated according to the sensing data. The analysis information is used to analyze the motion state.

Based on the above, the exercise analysis method, the server, and the terminal apparatus used for exercise analysis in the embodiment of the disclosure may transmit the collected sensing data to the server, analyze the motion state corresponding to the sensing data by the server, and present the analyzed motion state by the terminal apparatus. In this way, operational convenience and analysis efficiency may be improved.

To make the aforementioned more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate example embodiments of the disclosure and, together with the description, serve to explain the principles of the disclosure.

FIG. 1 is a component block diagram of an exercise system according to an embodiment of the disclosure.

FIG. 2A is a schematic diagram of a wearable device according to an embodiment of the disclosure.

FIG. 2B is a schematic diagram of a sensor and a relay device according to an embodiment of the disclosure.

FIG. 2C is a schematic diagram of a wearable device according to an embodiment of the disclosure.

FIG. 3 is a flow chart of an exercise analysis method according to an embodiment of the disclosure.

FIG. 4 is a schematic diagram of an exercise system according to an embodiment of the disclosure.

FIG. 5 is a schematic diagram illustrating a stance phase and a swing phase according to an embodiment of the disclosure.

FIG. 6A illustrates a relationship between a value of sensing data and time according to an embodiment of the disclosure.

FIG. 6B is a schematic diagram of analysis information according to an embodiment of the disclosure.

FIG. 6C is a schematic diagram of pressure center distribution according to an embodiment of the disclosure.

FIG. 7A to FIG. 7C are schematic diagrams illustrating a pressure center according to an embodiment of the disclosure.

FIG. 8A to FIG. 8D are schematic diagrams illustrating a trajectory of a pressure center according to an embodiment of the disclosure.

FIG. 9A is a pie chart of a stance phase and a swing phase according to an embodiment of the disclosure.

FIG. 9B is a bar chart of support time according to an embodiment of the disclosure.

### DESCRIPTION OF THE EMBODIMENTS

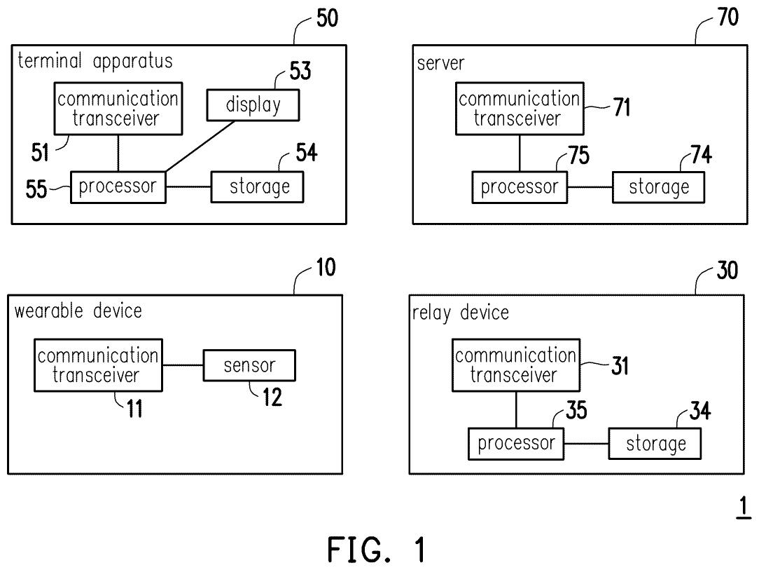

FIG. 1 is a component block diagram of an exercise system 1 according to an embodiment of the disclosure. Referring to FIG. 1, the exercise system 1 includes (but is not limited to) one or more wearable devices 10, one or more relay devices 30, one or more terminal apparatus 50, and one or more servers 70.

The wearable device 10 may be used for a device worn on a body part (e.g., head, chest, hand, or foot) or for a user to hold. For example, a smart watch, smart glasses, head-mounted display, handheld controller, game controller, somatosensory device, and ankle sensor.

The wearable device 10 includes (but is not limited to) a communication transceiver 11 and a sensor 12.

The communication transceiver 11 is, for example, a communication transceiver circuit that supports Bluetooth, Wi-Fi, mobile network, optical fiber network, or other communication technologies, or a transmission interface that supports USB, UART, or Thunderbolt. In one embodiment, the communication transceiver 11 is used to transmit signals to an external device (e.g., the relay device 30, the terminal apparatus 50, or the server 70). For example, data from the sensor 12 is transmitted. In some embodiments, the communication transceiver 11 is used to connect with an external device (e.g., the relay device 30 or the terminal apparatus 50) and transmit or receive data accordingly.

The sensor 12 is coupled to the communication transceiver 11. The sensor 12 may be an accelerometer, a gyroscope, a magnetic sensor, an inertial measurement unit (IMU), a multi-axis motion sensor, an image sensor, a force sensor, or an infrared sensor. In one embodiment, the sensor 12 is used to obtain sensing data. Values of the sensing data may correspond to intensity, angle, position, variation, and/or time points. The sensing data corresponds to a motion state. Depending on the design and/or application requirements, the type of the motion state may be walking, running, sitting, standing, shooting, or kicking, and is not limited thereto.

The relay device 30 includes (but is not limited to) a communication transceiver 31, a storage 34, and a processor 35.

The implementation and functions of the communication transceiver 31 can be referred to the description of the communication transceiver 11 and will not be repeated in the following. In one embodiment, the communication transceiver 31 is used to transmit or forward sensing data from the wearable device 10.

The storage 34 can be any type of fixed or removable random access memory (RAM), read only memory (ROM), flash memory, hard disk drive (HDD), solid-state drive (SSD), or similar components. In one embodiment, the storage 34 is used to store a program code, software module, configuration, data, or file (e.g., data, events, information, or features), as will be described in detail in subsequent embodiments.

The processor 35 is coupled to the communication transceiver 31 and the storage 34. The processor 35 may be a central processing unit (CPU), a graphics processing unit (GPU), or other programmable general-purpose or special-purpose microprocessor, digital signal processor (DSP), programmable controller, field programmable gate array (FPGA), application-specific integrated circuit (ASIC), neural network accelerator, or other similar components or combinations of the above components. In one embodiment, the processor 35 is used to execute all or part of the operations of the relay device 30, and may load and execute each program code, software module, file, and data stored in the storage 34.

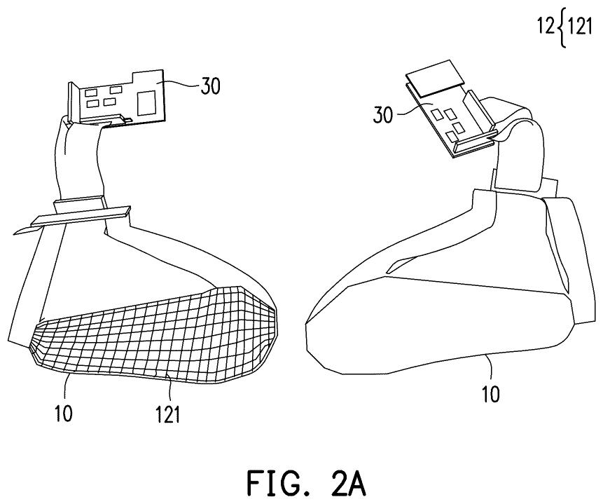

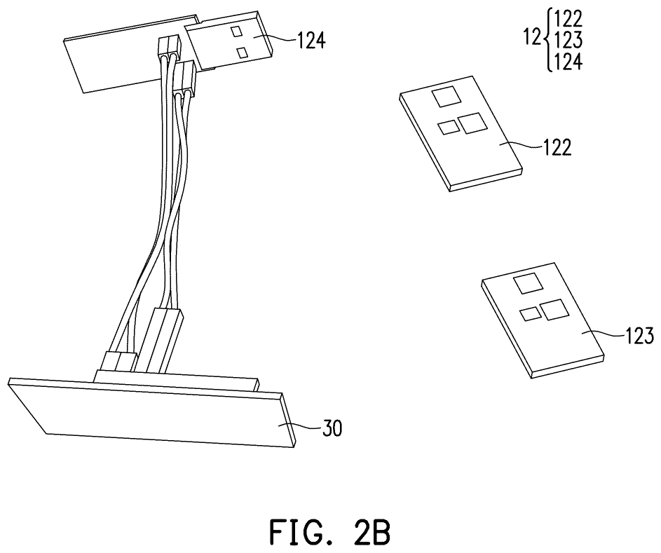

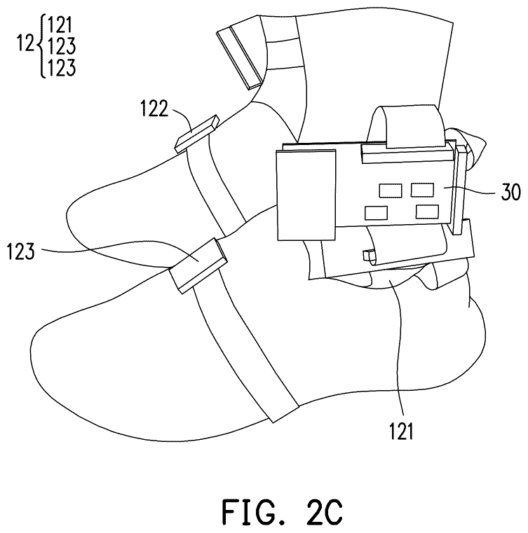

FIG. 2A is a schematic diagram of a wearable device 10 according to an embodiment of the disclosure. Referring to FIG. 2A, the wearable device 10 is a pressure sensing insole. The wearable device 10 includes an insole-type pressure sensor 121 (i.e., the sensor 12). The pressure sensor 121 includes, for example, electronic components such as an analog switch, a distributor, and an inverter. The pressure sensor 121 may obtain sensing data (e.g., pressure values) from multiple pressure sensing points and transmit the sensing data by the connected relay device 30.

FIG. 2B is a schematic diagram of sensors 122 and 123 and a relay device 30 according to an embodiment of the disclosure. Referring to FIG. 2B, IMUs 122 and 123 (i.e., the sensors 12) correspond to a left and right feet of a user respectively. A sensing receiver 124 is wirelessly connected to the IMUs 122 and 123, and is used to receive the sensing data (e.g., acceleration, angular velocity, and/or posture) of the IMUs 122 and 123. The sensing receiver 124 is wired to the relay device 30 so that the relay device 30 can transmit the sensing data from the IMUs 122 and 123.

FIG. 2C is a schematic diagram of a wearable device 10 according to an embodiment of the disclosure. Referring to FIG. 2C, the pressure sensor 121 can be placed in a shoe, and the IMUs 122 and 123 can be tied to the back of the shoe respectively. In addition, the relay device 30 is disposed next to an ankle.

It should be noted that FIG. 2A to FIG. 2C are only examples, and the user can adjust the components, quantity, and position according to actual needs.

Referring to FIG. 1, the terminal apparatus 50 may be a mobile phone, a tablet computer, a laptop computer, a desktop computer, a voice assistant device, a smart home appliance, a wearable device, a vehicle-mounted device, or other electronic devices.

The terminal apparatus 50 includes (but is not limited to) a communication transceiver 51, a display 53, a storage 54, and a processor 55.

Functions and implementation of the communication transceiver 51, the storage 54, and the processor 55 can be referred to the description of the communication transceiver 11, the storage 34, and the processor 35, and will not be repeated in the following.

In one embodiment, the communication transceiver 51 is used to receive the sensing data from the relay device 30 or the wearable device 10. In one embodiment, the communication transceiver 51 is used to transmit the sensing data from the relay device 30 or the wearable device 10.

The display 53 may be an LCD, LED display, OLED display, Mini LED display, or other types of displays. In one embodiment, the display 53 is used to display images, e.g., a user interface, videos, or photos.

The processor 55 is coupled to the communication transceiver 51, the display 53, and the storage 54. In one embodiment, the processor 55 is used to execute all or part of the operations of the terminal apparatus 50, and can load and execute each program code, software module, file, and data stored in the storage 54.

Referring to FIG. 1, the server 70 can be a cloud server, a computing server, a tablet computer, a laptop computer, a desktop computer, a voice assistant device, a smart home appliance, a wearable device, a vehicle-mounted device, or other electronic devices.

The terminal apparatus 70 includes (but is not limited to) a communication transceiver 71, a storage 74, and a processor 75.

Functions and implementation of the communication transceiver 71, the storage 74, and the processor 75 can be referred to the description of the communication transceiver 11, the storage 34, and the processor 35, and will not be repeated in the following.

In one embodiment, the communication transceiver 71 is used to receive data, e.g., the sensing data from the sensor 12, from the terminal apparatus 50. In one embodiment, the communication transceiver 71 is used to transmit data to the terminal apparatus 50.

The processor 75 is coupled to the communication transceiver 71 and the storage 74. In one embodiment, the processor 75 is used to execute all or part of the operations of the server 70, and can load and execute each program code, software module, file, and data stored in the storage 74.

In the following, the method described in the embodiment of the disclosed will be explained with reference to various apparatuses, components, and modules in the exercise system 1. Each process of the method can be adjusted according to the implementation situation, and is not limited thereto.

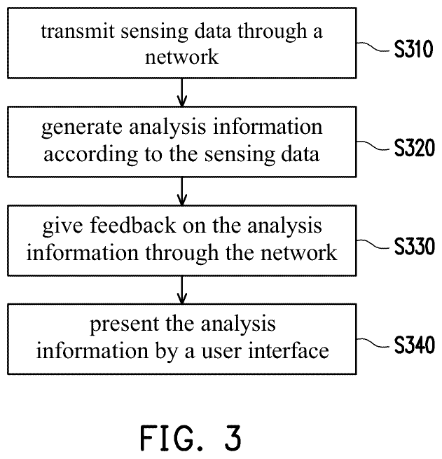

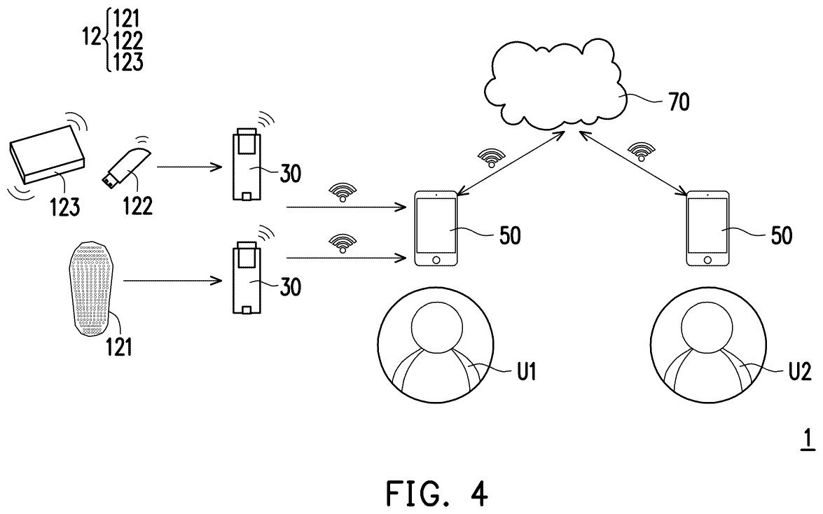

FIG. 3 is a flow chart of an exercise analysis method according to an embodiment of the disclosure. Referring to FIG. 3, the processor 55 transmits the sensing data through the network by the communication transceiver 51 (step S310). Specifically, FIG. 4 is a schematic diagram of an exercise system 1 according to an embodiment of the disclosure. Referring to FIG. 4, the relay device 30 obtains the sensing data of the sensor 12 such as the pressure sensor 121 and the IMUs 122 and 123. The sensing data may include identifying information to distinguish the type and/or source of the sensing data. The terminal apparatus 50 may be used by a user U1 (e.g., a trainer, patient, or other person) or a user U2 (e.g., a physician, physical therapist, or coach). After the relay device 30 is connected and paired with the terminal apparatus 50, the relay device 30 transmits the sensing data to the terminal apparatus 50. Then, the terminal apparatus 50 can transmit the sensing data to the server 70. The processor 75 can receive the sensing data through the communication transceiver 71.

In one embodiment, the network may be a local network, a private network, a public network, or the Internet.

In one embodiment, the processor 55 transmits the sensing data through WebSocket by the communication transceiver 51, and the processor 75 receives the sensing data through WebSocket by the communication transceiver 71. WebSocket is an application-level network transport protocol and is used for bidirectional data transfer. However, in other embodiments, other application-level network transport protocols may be used, for example, HTTP.

In one embodiment, the terminal apparatus 50 further transmits identity information to the server. The Identity information may include height, weight, age, or disease, or may include experience, certification, or expertise.

Referring to FIG. 3, the processor 75 generates analysis information according to the sensing data (step S320). Specifically, the analysis information is used to analyze a motion state, e.g., posture correctness, motion efficiency, or energy conversion. In one embodiment, the motion state includes multiple motion phases. The types of the phases vary depending on the application context.

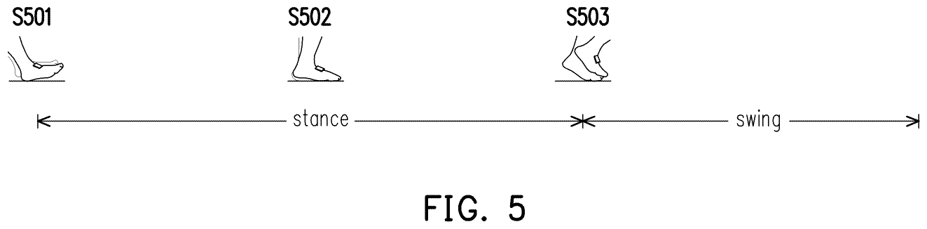

In an application scenario, the motion state is a walking posture (simply called gait). Gait analysis is the measurement of walking conditions by observation or scientific instruments, and the results of the observations and measurements are quantified into multiple indicators. FIG. 5 is a schematic diagram illustrating a stance phase and a swing phase according to an embodiment of the disclosure. Referring to FIG. 5, the indicators are usually based on three gait phases, which include the stance phase, the swing phase, and a two-legged stance phase (i.e., a motion phase). Each phase may correspond to one or more gait events, e.g., heel strike (step S501), heel off (step S502), and toe off (step S503). Steps S501 to S503 correspond to the stance phase, and steps S503 to S501 correspond to the swing phase. The stance phase and the swing phase with a single foot is often referred to as a stride.

In other application scenarios, the motion state is a state of running, swimming, pitching, or other motions.

In one embodiment, the processor 75 sorts the sensing data according to a time relationship. The time relationship is an ordering of multiple values in the sensing data corresponding to times points. The values are, for example, acceleration, angular velocity, angle, or intensity, but are not limited thereto. Each value corresponds to a time point. That is, one or more values are detected at a time point. The time relationship is the sequence of time points when the values are generated/obtained. The processor 75 may arrange the values according to the sequence of corresponding time points. For example, the earlier the time point is, the earlier the sorting is, and the later the time point is, the later the sorting is. Sorting multiple values helps to understand changes in sensing data over time.

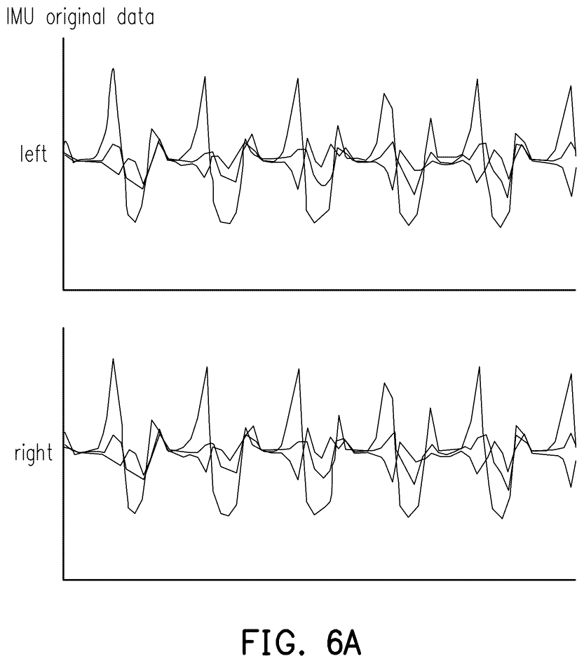

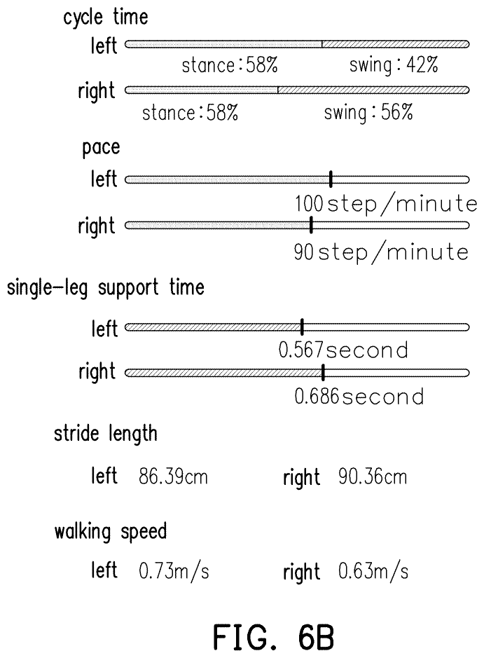

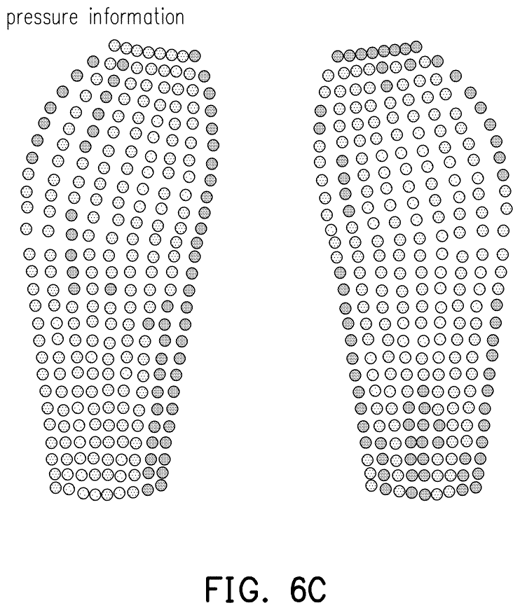

For example, FIG. 6A illustrates a relationship between a value of sensing data and time according to an embodiment of the disclosure. Referring to FIG. 6A, the values detected by the IMUs 122 and 123 change with time.

In one embodiment, the processor 75 may determine statistics for multiple motion phases. The analysis information includes the statistics for the motion phases. Taking gait analysis as an example, the statistics include, for example, double-leg support time (e.g., double-leg support time of each stride), cycle time (e.g., the proportion of time in the stance phase and swing phase of each stride), pace (e.g., number of steps taken per minute), single-leg support time (e.g., single-leg standing time per stride), stride length, walking speed, or pressure center of the left or right foot.

In one embodiment, the motion phase includes a stance phase and a swing phase. The processor 75 may identify whether the sensing data belongs to the stance phase or the swing phase. It can be seen from FIG. 5 that the gait events corresponding to the stance phase and the swing phase change with time. In addition, each gait event corresponds to a different posture or motion, and the posture or motion may be detected from the sensing data. Thus, posture events (e.g., gait events) may be estimated according to the values of the sensing data sorted based on time relationships, and the motion phases (e.g., the stance phase or the swing phase) corresponding to the values or statistics may be identified.

In one embodiment, the processor 75 may train a recognition model by a machine learning algorithm to understand the correlation between the values/statistics of the sensing data and the motion states. The machine learning algorithm is, for example, a convolutional neural network (R-CNN) or a generative adversarial network (GAN), but is not limited thereto. The trained recognition model may determine the motion state corresponding to the values/statistics of the sensing data. For example, the stance phase or the swing phase is determined according to the values of the sensing data of the IMUs 122 and 123 in FIG. 2B.

In one embodiment, the processor 75 may convert multiple values of the sensing data into analysis information. The analysis information is at least one of a statistic per unit time, duration, speed, and position distribution. The unit time may be one second, one minute, one hour, or one day, and is not limited thereto. The statistic may be cumulative times, averages, or sums, but is not limited thereto, e.g., the number of times the foot touches the ground in one minute. The duration is how long the same phase, state, and/or value is maintained, e.g., the duration of left and right foot touching the ground. The speed is, for example, walking, running, or swimming speed. At a time point, each value of the sensing data may correspond to a position. The position distribution presents corresponding values of multiple positions at a time point or a period. That is, a position corresponds to a value. For example, multiple pressure sensing points of the pressure sensor 121 in FIG. 2A respectively correspond to one position, and a pressure distribution diagram presents the pressure values corresponding to the pressure sensing points of the multiple positions.

In one embodiment, the value conversion may be performed using corresponding mathematical equations, lookup tables, and machine learning-based inferencers.

FIG. 6B is a schematic diagram of analysis information according to an embodiment of the disclosure. Referring to FIG. 6B, taking gait analysis as an example, the cycle time is the proportion of time in the stance phase and the swing phase of each stride, the pace is the number of steps taken in one minute, the single-leg support time is the time spent standing on one foot for each stride, the stride length is the distance of each stride of the left and right foot, and walking speed is the moving speed of the left and right foot.

In one embodiment, the position distribution is a pressure center distribution. Multiple values of the sensing data are multiple pressure values. The processor 75 may give corresponding weights according to the position. For example, the sensor 12 is provided with multiple pressure sensing points, and each pressure sensing point corresponds to a position and a weight. The weight of a certain pressure value is, for example, the proportion of the pressure value to the sum of all pressure values. The processor 75 may determine the pressure center in the pressure center distribution according to a weighted result of one or more pressure values and the corresponding weight. The weighted result is the sum of one or more pressure values multiplied by the corresponding weight. The pressure center is a representative position of one or more pressure values at a certain time point or a certain time interval, e.g., center position or center of gravity position.

FIG. 6C is a schematic diagram of pressure center distribution according to an embodiment of the disclosure. Referring to FIG. 6C, each circle corresponds to a pressure sensing point. The depth in the circle corresponds to the statistic size of the pressure center, where the deeper it is, the larger the statistic, and the shallower it is, the smaller the statistic. The pressure center distribution is a statistic of pressure centers at multiple sampling time points over a period of time (e.g., one minute or ten minutes), e.g., the number of times of the pressure center of 100 sampling time points located at the pressure sensing points within one minute.

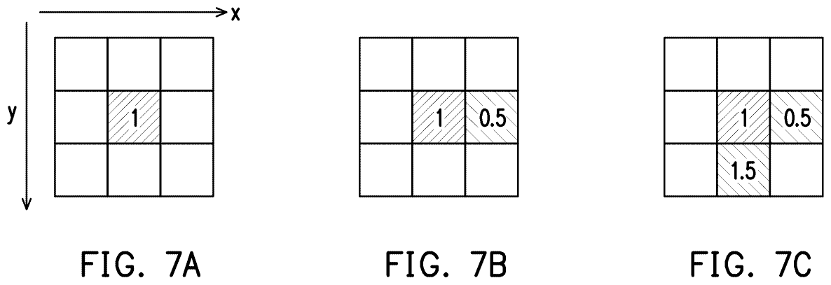

FIG. 7A to FIG. 7C are schematic diagrams illustrating a pressure center according to an embodiment of the disclosure. Referring to FIG. 7A, taking 3×3 pressure sensing points as an example, only one pressure sensing point (whose position is at coordinates (2,2)) has a pressure value (e.g., 1). Thus, the pressure center is located at coordinates (2,2).

Referring to FIG. 7B, there are two pressure sensing points with values, and their positions and pressure values are as follows: coordinates (2,2): 1; coordinates (3,2): 0.5. The weight is, for example, the proportion of one pressure value to the sum of all pressure values. The weight corresponding to coordinates (2,2) is 1/(1+0.5)=0.66, and the weight corresponding to coordinates (3,2) is 0.5/(1+0.5)=0.33. The mathematical expression of the pressure center is:

$$COP ( X , Y ) = [ \frac{W \times x}{\sum pressure value} , \frac{W \times y}{\sum pressure value} ] \qquad ( 1 )$$

where COP is the pressure center, the coordinates (X, Y) represent the horizontal axis coordinate is X and the vertical axis coordinate is Y, W is the weight, x is the horizontal axis coordinate of the pressure value, and y is the vertical axis coordinate of the pressure value. Thus, the weighted result of FIG. 7B is (2*0.66+3*0.33, 2*0.66+2*0.33)=(2.33, 2). That is, the pressure center is located at coordinates (2.33, 2).

Referring to FIG. 7B, there are three pressure sensing points with values, and their positions and pressure values are as follows: coordinates (2,2): 1; coordinates (3,2): 0.5; coordinates (2,3): 1.5. The weight corresponding to coordinates (2,2) is 1/(1+0.5+1.5)=0.33, the weight corresponding to coordinates (3,2) is 0.5/(1+0.5+1.5)=0.16, and the weight corresponding to coordinates (2,3) is 1.5/(1+0.5+1.5)=0.5. Thus, the pressure center is located at coordinates (2.17, 2.5).

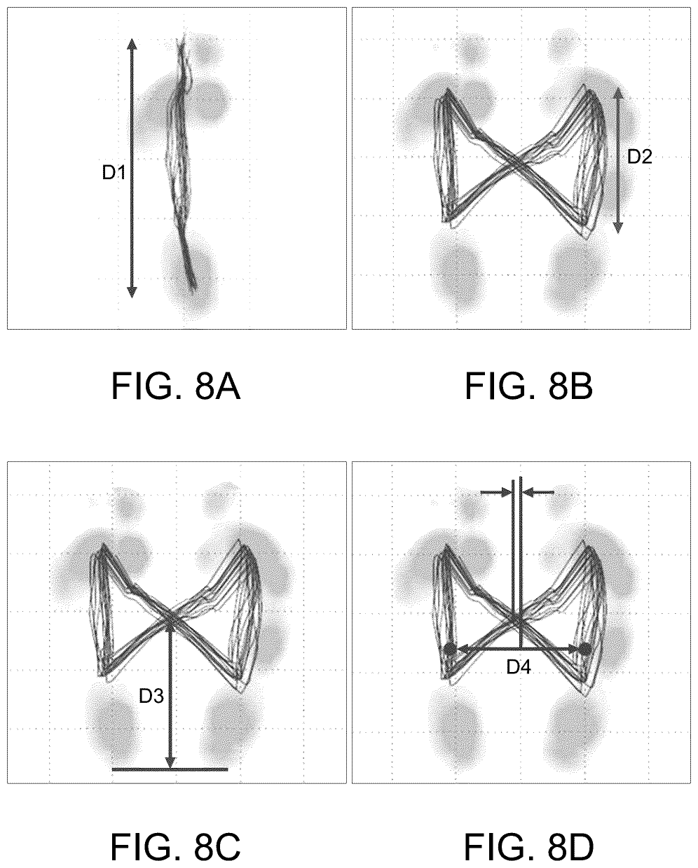

In one embodiment, the analysis information includes symmetry, e.g., the symmetry of the pressure center. The processor 75 may determine symmetry according to a trajectory of the pressure center at multiple time points. FIG. 8A to FIG. 8D are schematic diagrams illustrating a trajectory of a pressure center according to an embodiment of the disclosure. Referring to FIG. 8A, the trajectory reflects the pressure center changing its position over time. A length D1 is a length that the pressure center moves throughout the motion phase (e.g., gait phase). Referring to FIG. 8B, a length D2 is a motion length of the pressure center when supported by a single leg. Referring to FIG. 8C, a length D3 is a distance between the heels of both feet and a center intersection point of the trajectory of the pressure center. Referring to FIG. 8D, a length D4 is a horizontal distance from a center point of horizontal line of the trajectory connecting the pressure centers of the feet to the center intersection point.

In one embodiment, the mathematical expression of symmetry is:

$$2 \times \frac{left foot parameter - right foot parameter}{left foot parameter + right foot parameter} \times 100 \% \qquad ( 2 )$$

where the parameter can be walking speed, cycle time, stance/swing phase, support time, or stride length. Taking the symmetry of the walking speed of the left and right feet as an example, when the average speed of the left foot is 0.73 meters per second (m/s), the average speed of the right foot is 0.63 m/s. Thus, the symmetry index of the speed is 14.7%, which means that the left foot is faster than the right foot. If the average stride length of the right foot is 80 centimeters (cm) and the average stride length of the left foot is 10 cm, the symmetry index of the stride length is −155%, which means there is a large asymmetry in the stride length, e.g., within one stride, the right foot is 155% longer than the left foot.

Referring to FIG. 3, the processor 75 gives feedback/transmits analysis information through the network by the communication transceiver 71 (step S330). The processor 55 receives the analysis information through the network by the communication transceiver 51. Taking FIG. 4 as an example, the terminal apparatus 50 of users U1 and U2 obtains the analysis information from the server 70.

In one embodiment, the server 70 transmits analysis data to the terminal apparatus 50 through WebSocket.

Referring to FIG. 3, the processor 55 presents the analysis information through the user interface by the display 53 (step S340). Specifically, the user interface may present text, numbers, symbols, charts, graphics, photos, or videos. For example, the user interface presents FIG. 6A to FIG. 6C and FIG. 8A to FIG. 8D.

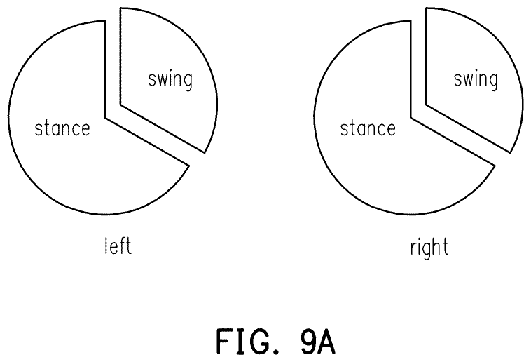

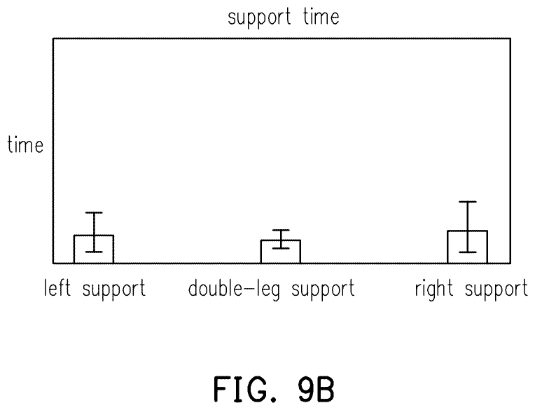

In addition to being expressed in text, it can also be converted into other charts. As another example, FIG. 9A is a pie chart of a stance phase and a swing phase according to an embodiment of the disclosure. Referring to FIG. 9A, the pie chart can show the difference in the time proportion of the stance phase and the swing phase of each stride, e.g., the stance phase is longer. The user interface can be rendered as shown in FIG. 9A.

As another example, FIG. 9B is a bar chart of support time according to an embodiment of the disclosure. Referring to FIG. 9B, the bar chart can show the difference in the support time of the left foot, the right foot, and both feet, e.g., the support time of the left foot and the right foot is longer. This provides an intuitive and simple interface.

In an application scenario, multiple types of analysis information can be integrated into analysis reports. The analysis report can be used by the user U1 in FIG. 3 to adjust or improve exercise or rehabilitation, and can be used by the user U2 in FIG. 3 to diagnose and analyze or adjust the training plan.

In an application scenario, an application program of the terminal apparatus 50 can provide a reservation function, allowing the users U1 and U2 to schedule a time to discuss analysis information/reports. In another application scenario, the analysis information can be used for knowledge exploration or new discoveries in scientific research and other fields.

To sum up, in the exercise analysis method, the server and the terminal apparatus used for exercise analysis in the embodiment of the disclosure, the sensing data can be uploaded to the server, the corresponding analysis information can be generated by the server, and the analysis information can be presented by the terminal apparatus. In this way, multi-sensing data may be integrated, data may be centrally managed, user-friendly operation may be improved, training efficiency may be improved, user experience may be improved, and analysis indicators may be standardized.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. An exercise analysis method, comprising:
transmitting sensing data through a network, wherein the sensing data corresponds to a motion state;
generating analysis information according to the sensing data, wherein the analysis information is used to analyze the motion state;
giving feedback on the analysis information through the network; and
presenting the analysis information by a user interface.

2. The exercise analysis method according to claim 1, wherein generating the analysis information according to the sensing data comprises:
sorting the sensing data according to a time relationship, wherein the time relationship is an ordering of a plurality of values in the sensing data corresponding to times points, and the analysis information comprises the sorted values.

3. The exercise analysis method according to claim 1, wherein generating the analysis information according to the sensing data comprises:
converting a plurality of values in the sensing data into the analysis information, wherein the analysis information is at least one of a statistic per unit time, a duration, a speed, and a position distribution.

4. The exercise analysis method according to claim 3, wherein the position distribution is a pressure center distribution, the values are a plurality of pressure values, and converting the values into the analysis information comprises:
giving corresponding weights to the pressure values according to position; and
determining a pressure center in the pressure center distribution according to a weighted result of at least one of the pressure values and the corresponding weight.

5. The exercise analysis method according to claim 4, comprising:
determining symmetry according to a trajectory of the pressure center at a plurality of time points, wherein the analysis information comprises the symmetry.

6. The exercise analysis method according to claim 1, wherein the motion state comprises a plurality of motion phases, and generating the analysis information according to the sensing data comprises:
determining a statistic of the motion phases, wherein the analysis information comprises the statistic of the motion phases.

7. The exercise analysis method according to claim 6, wherein the motion phases comprise a stance phase and a swing phase, and the exercise analysis method further comprises:
identifying whether the sensing data belongs to the stance phase or the swing phase.

8. The exercise analysis method according to claim 1, wherein the motion state is a walking state.

9. The exercise analysis method according to claim 1, wherein transmitting the sensing data through the network comprises:
transmitting the sensing data by WebSocket.

10. A server used for exercise analysis, comprising:
a communication transceiver;
a storage, storing a program code; and
a processor, coupled to the communication transceiver and the storage, loading the program code and execute:
receiving sensing data through a network by the communication transceiver, wherein the sensing data corresponds to a motion state;
generating analysis information according to the sensing data, wherein the analysis information is used to analyze the motion state; and
giving feedback on the analysis information through the network by the communication transceiver, wherein the analysis information is used to be presented by a user interface.

11. The server used for exercise analysis according to claim 10, wherein the processor further executes:
sorting the sensing data according to a time relationship, wherein the time relationship is an ordering of a plurality of values in the sensing data corresponding to times points, and the analysis information comprises the sorted values.

12. The server used for exercise analysis according to claim 10, wherein the processor further executes:
converting a plurality of values in the sensing data into the analysis information, wherein the analysis information is at least one of a statistic per unit time, a duration, a speed, and a position distribution.

13. The server used for exercise analysis according to claim 12, wherein the position distribution is a pressure center distribution, the values are a plurality of pressure values, and the processor further executes:
giving corresponding weights to the pressure values according to position; and
determining a pressure center in the pressure center distribution according to a weighted result of at least one of the pressure values and the corresponding weight.

14. The server used for exercise analysis according to claim 13, wherein the processor further executes:
determining symmetry according to a trajectory of the pressure center at a plurality of time points, wherein the analysis information comprises the symmetry.

15. The server used for exercise analysis according to claim 10, wherein the motion state comprises a plurality of motion phases, and the processor further executes:
determining a statistic of the motion phases, wherein the analysis information comprises the statistic of the motion phases.

16. The server used for exercise analysis according to claim 15, wherein the motion phases comprise a stance phase and a swing phase, and the processor further executes:
identifying whether the sensing data belongs to the stance phase or the swing phase.

17. The server used for exercise analysis according to claim 10, wherein the motion state is a walking state.

18. The server used for exercise analysis according to claim 10, wherein the processor further executes:
receiving the sensing data through WebSocket by the communication transceiver.

19. A terminal apparatus used for exercise analysis, comprising:
a display;
a communication transceiver;
a storage, storing a program code; and
a processor, coupled to the display, the communication transceiver, and the storage, loading the program code and executes:
transmitting sensing data through a network by the communication transceiver, wherein the sensing data corresponds to a motion state;
receiving analysis information through the network by the communication transceiver, wherein the analysis information is generated according to the sensing data, and the analysis information is used to analyze the motion state; and
presenting the analysis information on a user interface by the display.

20. The terminal apparatus used for exercise analysis according to claim 19, wherein the motion state is a walking state.
